(19) United States
(12) Patent Application Publication
(10) Pub. No.: US 2025/0195718 A1
(43) Pub. Date: Jun. 19, 2025

(54) KAOLIN-BASED COMPOSITE HEMOSTATIC POWDER AND PREPARATION METHOD THEREOF

(71) Applicant: CHINA UNIVERSITY OF GEOSCIENCES (WUHAN), Wuhan (CN)

(72) Inventors: Huaming YANG, Wuhan (CN); Zhiyong ZENG, Wuhan (CN); Hao WANG, Wuhan (CN)

(73) Assignee: CHINA UNIVERSITY OF GEOSCIENCES (WUHAN), Wuhan (CN)

(21) Appl. No.: 19/065,113

(22) Filed: Feb. 27, 2025

(30) Foreign Application Priority Data
Mar. 25, 2024 (CN) 202410342097.8

(51) Int. Cl. A61L 26/00

(52) U.S. Cl. CPC A61L 26/0095; A61L 26/0066; A61L 2300/102; A61L 2300/418; A61L 2400/04

(57) ABSTRACT

The present disclosure discloses a kaolin-based composite hemostatic powder and a preparation method thereof. The present disclosure relates to the technical field of hemostatic powders. The kaolin-based composite hemostatic powder is gelatin-modified kaolin. The preparation method includes: thoroughly mixing a gelatin and kaolin in a solvent to produce a mixed solution, lyophilizing the mixed solution to remove the solvent, and grinding into a powder to produce the kaolin-based composite hemostatic powder. The prepared gelatin-modified kaolin hemostatic powder exhibits excellent effects in skin adhesion, in vitro hemostasis, and hemocompatibility experiments, and breaks the limitations of the previous kaolin powders in hemostasis.

### CROSS-REFERENCE TO RELATED APPLICATIONS

This application claims priority to Chinese Patent Application No. 202410342097.8 with a filing date of Mar. 25, 2024. The content of the aforementioned application, including any intervening amendments thereto, is incorporated herein by reference.

### TECHNICAL FIELD

The present disclosure relates to the technical field of hemostatic powders, and in particular to a kaolin-based composite hemostatic powder and a preparation method thereof.

### BACKGROUND

Efficient hemostatic materials are the key materials for first aid for trauma in major sudden traffic accidents, wars, natural disasters, etc. The uncontrolled bleeding caused by severe trauma is associated with the high mortality. Kaolin (Al₂Si₂O₅(OH)₄) is a 1:1 layered aluminosilicate nanoclay, which is composed of tetrahedral SiO₄ sheets and octahedral Al(OH)₃ sheets stacked on the tetrahedral SiO₄ sheets. Kaolin has a typical two-dimensional sheet structure, a negatively-charged surface, and high hydrophilicity. Due to characteristics such as high biosafety, surface negative charges, and high hydrophilicity, kaolin has become an effective hemostatic material. The current kaolin-based hemostatic products on the market are mainly bulk materials such as gauzes or dressings, including QuickClot Combat Gauze, First-Aid Kaolin Hemostatic Dressing, QuickClot Sponge, etc. In addition, the Chinese patent CN115463242A discloses a kaolin hemostatic gauze and a preparation method thereof. The kaolin hemostatic gauze is prepared through steps such as loading α-Fe₂O₃-loaded kaolin on a medical nonwoven fabric, doping Ce, pressing, and impregnation. The kaolin hemostatic gauze prepared by this method has excellent biocompatibility. However, the simple impregnation alone can hardly make kaolin tightly adhere to a gauze, and the kaolin powder is easy to get rid of the gauze, which will reduce the hemostatic effect. The Chinese patent CN215689047U discloses a design of a kaolin hemostatic gauze. Kaolin is attached to both the front and back sides of a main body of the kaolin hemostatic gauze, and the kaolin is adopted as a hemostatic agent. However, the kaolin hemostatic gauze is merely suitable for superficial wounds with shallow bleeding points, and exhibits low hemostasis efficiency for deep bleeding point-based wounds and irregular wounds. Therefore, there is an urgent need to develop new forms of kaolin hemostatic products.

A powdery hemostatic material can enter a wound to act on deep bleeding points, and can also effectively cover an irregular bleeding wound, thereby improving the hemostasis efficiency. The current commercially-available powdery hemostatic materials include zeolite-based QuickClot, chitosan-based Celox, and starch-based Arista, for example. There are currently no products related to powdery kaolin-based hemostatic materials on the market. The zeolite hemostatic powder undergoes an exothermic phenomenon when in use, which will cause burning to normal tissues. The chitosan and starch hemostatic powders based on natural polymer materials usually exhibit a weak mechanical strength. Studies have confirmed that kaolin-based hemostatic materials and products undergo no exothermic phenomenon in a hemostatic process, which can avoid the burning to tissues. The rigid structure of kaolin itself can improve the mechanical strength of a material, but lacks the tissue adhesion. A hemostatic powder prepared directly from kaolin is easy to fall off under the action of a fast or high-pressure blood flow, which brings the risk of thrombosis and is even life-threatening. Therefore, kaolin cannot be used directly as a hemostatic powder.

### SUMMARY OF PRESENT INVENTION

An objective of the present disclosure: In view of the above-mentioned deficiencies of the prior art, the present disclosure provides a kaolin-based composite hemostatic powder that can be easily produced on a large scale with a simple process and easy operations, and a preparation method of the kaolin-based composite hemostatic powder. The kaolin-based composite hemostatic powder can be used for biomedicine. The kaolin-based composite hemostatic powder can improve the tissue adhesion, biocompatibility, and in vitro hemostatic performance of kaolin.

The present disclosure provides a kaolin-based composite hemostatic powder, where the kaolin-based composite hemostatic powder is gelatin-modified kaolin.

The present disclosure also provides a preparation method of the kaolin-based composite hemostatic powder described above, including: thoroughly mixing a gelatin and kaolin in a solvent to produce a mixed solution, lyophilizing the mixed solution to remove the solvent, and grinding into a powder to produce the kaolin-based composite hemostatic powder.

Further, a mass ratio of the gelatin to the kaolin is (2-1):(1-7).

Further, a mass ratio of the gelatin to the kaolin is (2-1):(1-4).

Further, the solvent is water.

Further, the mixing is conducted at 50° C. to 60° C.

Further, the mixing is conducted for at least 1 h.

Further, the preparation method includes: dissolving the gelatin in the water at 60° C. to produce a homogeneous gelatin solution, slowly adding a kaolin solution with a specified mass concentration dropwise to the homogeneous gelatin solution, and thoroughly mixing.

Further, the mass concentration of the kaolin solution is 5 wt % to 20 wt %.

Further, the lyophilizing is conducted at −30° C. to −20° C. for 2 d to 3 d.

In the present disclosure, kaolin is compounded with a gelatin, such that the advantages of the inorganic material and the biological polymer material are integrated. With kaolin as a raw material and a gelatin as a modifier, the present disclosure prepares a hemostatic powder with excellent tissue adhesion and biocompatibility through lyophilization and grinding, and improves the hemostatic performance of the kaolin powder. When adhering to a bleeding wound, the kaolin-based composite hemostatic powder can absorb water from the wound blood to concentrate blood cells and platelets, thereby stimulating the coagulation cascade and promoting the rapid hemostasis at the bleeding wound. The prepared gelatin-modified kaolin hemostatic powder exhibits excellent effects in skin adhesion, in vitro hemostasis, and hemocompatibility experiments, and breaks the limitations of the previous kaolin powders in hemostasis.

The gelatin adopted by the present disclosure has temperature sensitivity and excellent biocompatibility. At a temperature lower than 37° C., the gelatin can form gel blocks and maintain structural stability, which can avoid the diffusion of kaolin powder particles. A molecular chain of the gelatin includes a large number of carboxyl, amino, and hydroxyl functional groups. Thus, macromolecular chains of the gelatin can form a helical structure to cause gelation. When a temperature is higher than 37° C., with the increase of the temperature, the helical structure of macromolecular chains of the gelatin is gradually unraveled, which promotes the rapid absorption of water and improves the biocompatibility of kaolin. In addition, the large number of amino, carboxyl, and hydroxyl functional groups on the macromolecular chains of the gelatin can interact with amino groups on the skin surface to form hydrogen bonds, which improves the tissue adhesion of the kaolin hemostatic powder and solves the problem that kaolin cannot be directly used as a hemostatic powder.

With the gelatin and kaolin that have abundant reserves and low prices as raw materials, the present disclosure prepares a gelatin-modified kaolin hemostatic powder, which improves the hemostatic performance and biocompatibility of kaolin and is conducive to the high-value utilization of kaolin mineral materials.

Only two raw materials are adopted in the present disclosure. The kaolin has abundant reserves and a low price. The gelatin is non-toxic and easily-available. The preparation method of the present disclosure involves a simple and eco-friendly process without special protection, does not have high requirements for devices, has strong operability, and is conducive to large-scale production. In addition, in the preparation method, there is no need to use organic solvents and toxic and harmful chemicals, resulting in environmental friendliness.

### BRIEF DESCRIPTION OF THE DRAWINGS

FIG. 1 shows an infrared spectrum of a gelatin-modified kaolin (mass ratio: 1:1) prepared in Example 1;

FIG. 2 shows results of a powder-gel conversion experiment for gelatin-modified kaolin products;

FIG. 3 shows results of a skin adhesion experiment for a gelatin-modified kaolin powder;

FIG. 4 shows results of in vitro coagulation times of gelatin-modified kaolin powders;

FIG. 5 shows results of a coagulation index experiment for gelatin-modified kaolin powders; and

FIG. 6 shows results of a hemocompatibility experiment for gelatin-modified kaolin powders.

### DETAILED DESCRIPTION OF THE EMBODIMENTS

The technical solutions of the present disclosure are described in further detail below with reference to the specific embodiments and accompanying drawings, but the present disclosure is not limited thereto.

### EXAMPLE 1

A powder preparation method for improving the hemostatic performance of kaolin was provided, including the following step: specified amounts of a gelatin and kaolin were thoroughly mixed. Specifically, the gelatin was dissolved at 60° C. to produce a homogeneous gelatin solution, then a kaolin solution with a specific mass concentration was slowly added dropwise to the homogeneous gelatin solution, a reaction was conducted for 2 h to produce a reaction solution, and then the reaction solution was lyophilized and ground into a powder to obtain a product denoted as G/Kaolin for later use.

The product was prepared according to the following mass ratios of the gelatin to the kaolin: 2:1, 1:1, 1:2, 1:5, and 1:7. Experiments were conducted to prepare the powder according to the above method. Three clay minerals were adopted in experiments, namely, kaolin, montmorillonite, and bentonite. The experiments were conducted under the same experimental conditions except for different clay minerals.

### Experimental Testing

In this embodiment, the powder-gel conversion, skin adhesion, and hemostatic performance of the gelatin-modified kaolin hemostatic powder were verified.

### Skin Adhesion Experiment

The skin adhesion experiment was specifically conducted according to the following steps: An equal volume of deionized water was added to each of gelatin-modified kaolin hemostatic powders prepared according to different mass ratios, and photographing was conducted for recording. For the skin adhesion experiment, a 2×8 cm pig skin was taken to simulate the human skin tissue. A specific volume of a gelatin-modified kaolin hemostatic powder solution was taken and placed on the pig skin tissue, and observed and photographed to record an adhesion effect of a gelatin-modified kaolin hemostatic powder to the pig skin tissue.

### In Vitro Hemostasis Experiment (Coagulation Index Experiment)

10 mg of a sample was weighed and added to a 2 mL centrifuge tube, then 100 μL of anticoagulated rabbit blood was added, and then 10 μL of a 0.1 M CaCl₂ solution was immediately added to produce a mixed system. The mixed system was incubated at 37° C. for 5 min, and then 2 mL of deionized water was slowly added. A resulting supernatant liquid was then collected and centrifuged (3,000 rpm, 3 min) for separation. A centrifugal supernatant of each group was collected and tested for absorbance by a microplate reader at 540 nm. Deionized water was adopted as a blank control group. 3 replicates were set for each sample.

### In Vitro Coagulation Time Experiment

10 mg of a sample was added to a 2 mL centrifuge tube, then 100 μL of anticoagulated rabbit blood was added, and then 10 μL of a 0.1 M CaCl₂ solution was immediately added to produce a mixed system. The mixed system was incubated at 37° C. The centrifuge tube was inverted every 20 s to observe whether clots were formed, and a coagulation time was recorded. 3 replicates were set for each sample.

### Biocompatibility Experiment

A specified amount of whole blood was collected, centrifuged, and washed 3 times with phosphate buffered saline (PBS) to collect blood cells. Then 10 mg of a sample was added to a 2 mL centrifuge tube, and the collected blood cells were added to the centrifuge tube to produce a mixed system. Deionized water was adopted as a positive control group, and PBS was adopted as a negative control group. The mixed system was incubated at 37° C. for 1 h. Then a resulting supernatant liquid of each group was collected and tested for absorbance by a microplate reader at 540 nm. 3 replicates were set for each sample.

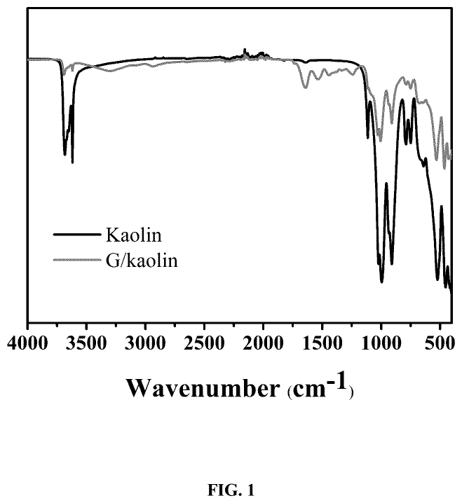

As shown in FIG. 1, an infrared spectrum of the gelatin-modified kaolin (mass ratio: 1:1) prepared in Example 1 showed that, after the gelatin modification, obvious absorption peaks of an amide I band and an amide II band appeared at 1,529 cm⁻¹ and 1,653 cm⁻¹, respectively, indicating the successful compounding of the gelatin with the kaolin.

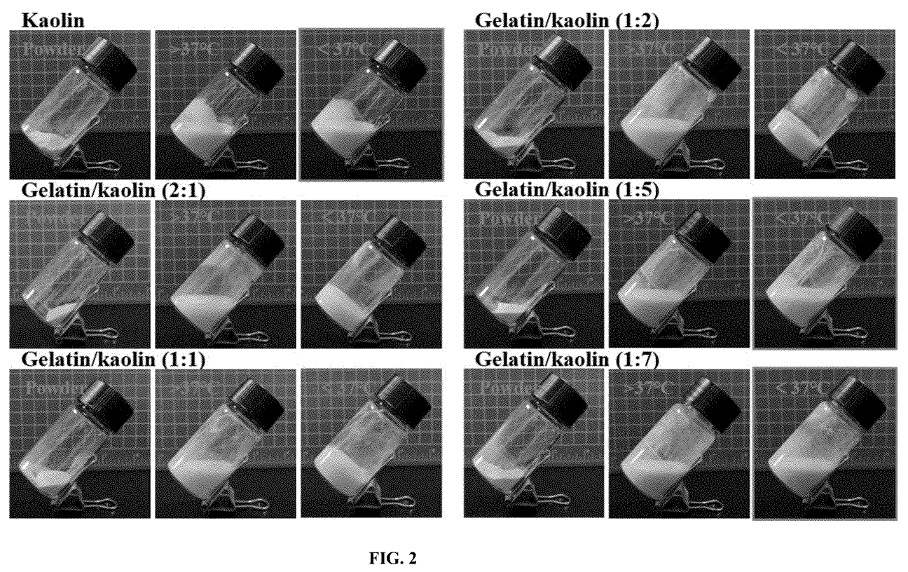

As shown in FIG. 2, the prepared gelatin-modified kaolin products each were subjected to a powder-gel conversion experiment. Experimental results showed that the pure kaolin powder and gelatin-modified kaolin powders with mass ratios of 1:5 and 1:7 could not undergo gelation (marked by red borders in FIG. 2), and gelatin-modified kaolin powders with mass ratios of 2:1, 1:1, and 1:2 could undergo gelation and adhere to a bottom of a glass bottle. In addition, when a temperature was higher than 37° C., macromolecule chains of the gelatin were uncoiled to form a solution state, indicating a temperature-responsive behavior.

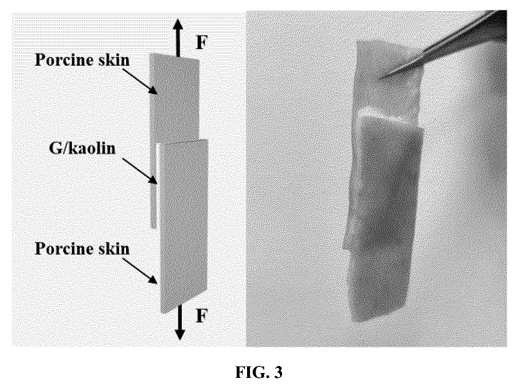

As shown in FIG. 3, the prepared gelatin-modified kaolin powder was subjected to a skin adhesion experiment. It can be seen from FIG. 3 that the gelatin-modified kaolin powder firmly adhered to the pig skin tissue, indicating that the gelatin-modified kaolin powder exhibited excellent skin tissue adhesion.

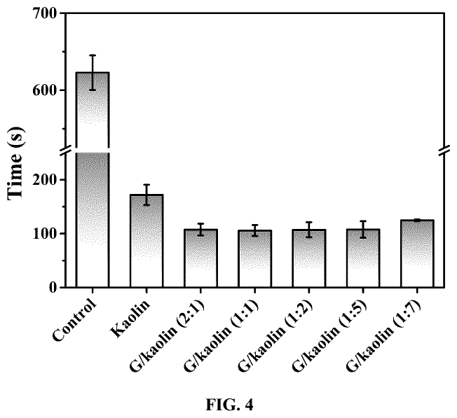

As shown in FIG. 4, the prepared gelatin-modified kaolin powders each were subjected to an in vitro coagulation time experiment. It can be seen from this figure that in vitro coagulation times of the control group and the kaolin group were 622±22 s and 171±19 s, respectively, and after the gelatin modification, coagulation times of gelatin-modified kaolin powder groups with mass ratios of 2:1, 1:1, 1:2, 1:5, and 1:7 were 107±19 s, 105±10 s, 107±14 s, 108±15 s, and 124±3 s, respectively. The experimental results indicated that the gelatin could improve an in vitro hemostatic effect of the kaolin.

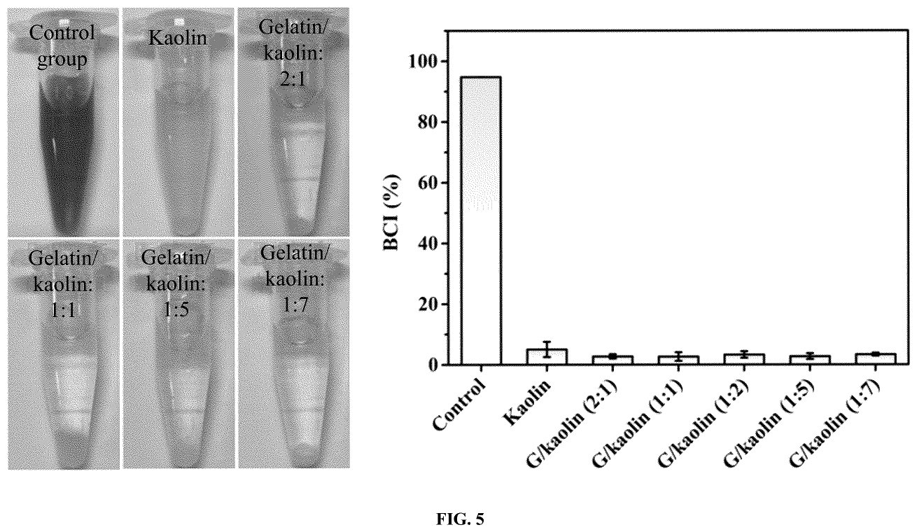

As shown in FIG. 5, the prepared gelatin-modified kaolin powders each were subjected to a coagulation index experiment. It can be seen from the experimental results in this figure that, within 5 min, there were no blood clots, a large amount of hemoglobin dissolved in water, and a bright-red supernatant liquid in the control group, but there was a transparent supernatant liquid in other groups (FIG. 5a). A supernatant liquid of each group was tested for absorbance by a microplate reader at 540 nm. According to results in FIG. 5b, a coagulation index in the control group was the highest, while coagulation indexes in other groups were relatively low, indicating a relatively-high hemoglobin content in the control group and relatively-low hemoglobin contents in other experimental groups, which were consistent with the experimental results in FIG. 5a. This experiment further indicated that the gelatin-modified kaolin powders had excellent in vitro hemostatic effects.

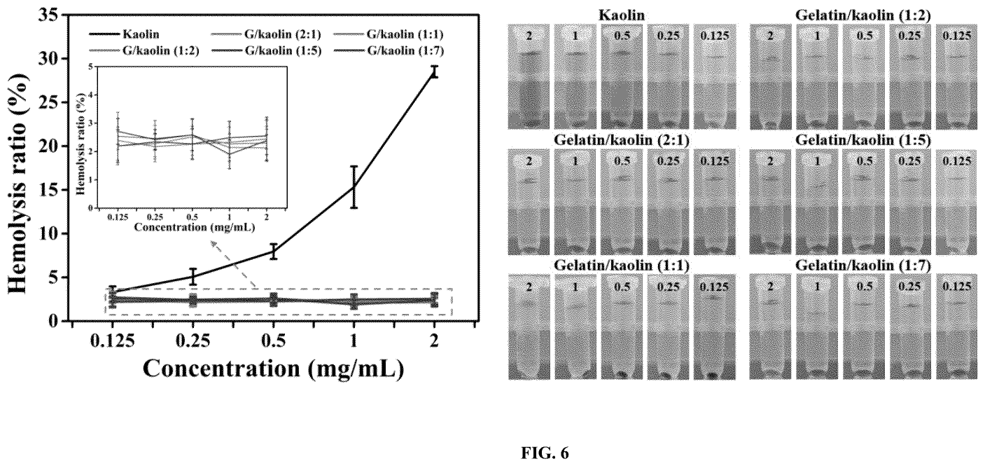

As shown in FIG. 6, the prepared gelatin-modified kaolin powders each were subjected to a hemocompatibility experiment. As shown in FIG. 6a, the pure kaolin powder has a high hemolysis rate. At a concentration of 2 mg/mL, the pure kaolin powder has a hemolysis rate of 28.5%. With the decrease of a kaolin concentration, the hemolysis rate of the pure kaolin powder is further reduced. At a concentration of 0.125 mg/mL, the pure kaolin powder has a hemolysis rate of 3.3%. However, after the gelatin modification, the gelatin-modified kaolin powder groups with mass ratios of 2:1, 1:1, 1:2, 1:5, and 1:7 all had a hemolysis rate of less than 5% at a concentration of either 2 mg/mL or 0.125 mg/mL. According to the pictures of experimental groups in FIG. 6b: The high-concentration kaolin led to an obvious hemolysis phenomenon, and with the decrease of a concentration, the hemolysis phenomenon was significantly improved. However, there was no obvious hemolysis phenomenon in other experimental groups, indicating that the gelatin-modified kaolin had excellent biocompatibility.

What is not mentioned above can be acquired in the prior art.

Although some specific embodiments of the present disclosure have been described in detail by way of examples, those skilled in the art will appreciate that the above examples are provided for illustration only and not for limiting the scope of the present disclosure. A person skilled in the art can make various modifications or supplements to the specific embodiments described or replace them in a similar manner, but it may not depart from the direction of the present disclosure or the scope defined by the appended claims. Those skilled in the art should understand that any modification, equivalent replacement, and improvement made to the above embodiments according to the technical essence of the present disclosure shall be included in the protection scope of the present disclosure.

What is claimed is:

1. A kaolin-based composite hemostatic powder, wherein the kaolin-based composite hemostatic powder is powder of gelatin-modified kaolin.

2. A preparation method of the kaolin-based composite hemostatic powder according to claim 1, comprising:
thoroughly mixing a gelatin and kaolin in a solvent to produce a mixed solution,
lyophilizing the mixed solution to remove the solvent, and
grinding into a powder to produce the kaolin-based composite hemostatic powder.

3. The preparation method according to claim 2, wherein a mass ratio of the gelatin to the kaolin is (2-1):(1-7).

4. The preparation method according to claim 2, wherein a mass ratio of the gelatin to the kaolin is (2-1):(1-4).

5. The preparation method according to claim 2, wherein the solvent is water.

6. The preparation method according to claim 5, wherein the mixing is conducted at 50° C. to 60° C.

7. The preparation method according to claim 5, wherein the mixing is conducted for at least 1 h.

8. The preparation method according to claim 2, wherein the thoroughly mixing the gelatin and the kaolin in the solvent to produce the mixed solution comprises:
dissolving the gelatin in water at 50° C. to 60° C. to produce a homogeneous gelatin solution,
slowly adding a kaolin solution with a specified mass concentration dropwise to the homogeneous gelatin solution, and
thoroughly mixing.

9. The preparation method according to claim 8, wherein the mass concentration of the kaolin solution is 5 wt % to 20 wt %.

10. The preparation method according to claim 8, wherein the lyophilizing is conducted at −30° C. to −20° C. for 2 d to 3 d.
